(19) United States
(12) Patent Application Publication
(10) Pub. No.: US 2025/0097649 A1
(43) Pub. Date: Mar. 20, 2025

(54) HEARING SYSTEM, AND METHOD FOR PRODUCING MONAURAL OR BINAURAL BEATS

(71) Applicant: Sivantos Pte. Ltd., Singapore (SG)

(72) Inventors: Matthias FRÖHLICH, Erlangen (DE); Jens HAIN, Erlangen (DE); Florian HÖGL, Erlangen (DE); Frank NAUMANN, Erlangen (DE)

(21) Appl. No.: 18/889,653

(22) Filed: Sep. 19, 2024

(30) Foreign Application Priority Data
Sep. 19, 2023 (DE) 10 2023 209 095.3

(51) Int. Cl. H04R 25/00; H03F 3/181

(52) U.S. Cl. CPC H04R 25/552; H04R 25/505; H03F 3/181; H03F 2200/03; H04R 2430/01

(57) ABSTRACT

A hearing system in particular a binaural hearing system that is configured to produce monaural or binaural beats and, to that end, has at least one hearing device containing a primary signal path. The hearing device moreover has a secondary signal path, containing a signal generator for generating a first beat signal. The first beat signal being frequency-shifted in relation to a further signal by a beat frequency in such a way that the effect of the monaural or binaural beat is produced for the user during operation. The signal generator has an associated adjusting apparatus that has an adjustable amplifier element. The adjusting apparatus has an analysis unit for determining an instantaneous signal level of the input signal, and the respective adjusting apparatus is configured to set a signal level of the first beat signal on the basis of the instantaneous signal level.

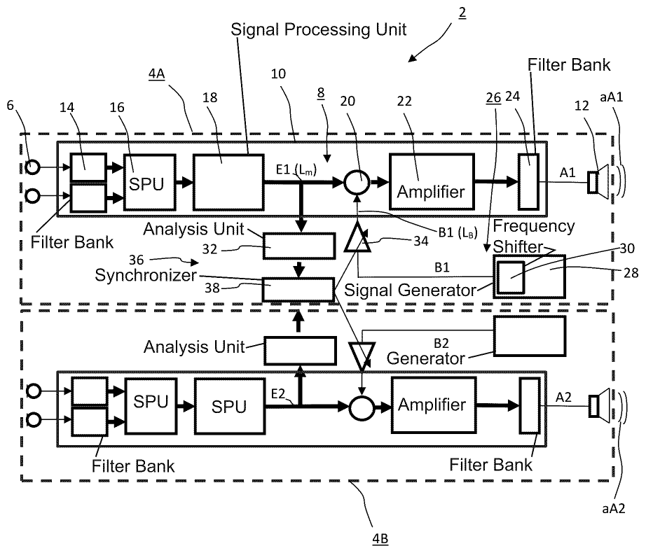

### CROSS-REFERENCE TO RELATED APPLICATION

This application claims the priority, under 35 U.S.C. § 119, of German Patent Application DE 10 2023 209 095.3, filed Sep. 19, 2023; the prior application is herewith incorporated by reference in its entirety.

### FIELD AND BACKGROUND OF THE INVENTION

The invention relates to an in particular binaural hearing system and to a method for producing monaural or binaural beats using such a hearing system.

Hearing system in the present case is understood to mean a system containing at least one hearing device and, in the case of a binaural hearing system, a system containing two hearing devices. The hearing devices are electronic devices that a user wears on the ear, behind the ear or in the ear. The hearing system is in particular a hearing aid system that is configured to compensate for a user-specific hearing impairment in a person with impaired hearing.

In each instance, such a hearing device normally has an input transducer that first converts an acoustic or streamed input signal into an electrical input signal, which is subsequently conditioned by an in particular digital signal processing unit to form an electrical output signal, which is routed to an output transducer that converts the electrical output signal into an acoustic output signal, which is delivered to the ear of the wearer.

Monaural beats or binaural beats are understood to mean known effects of a sensory perception in the human brain that involves the user perceiving a pulsating tone (beat) if they are presented with two identical acoustic signals having slightly differing frequencies. In the case of binaural beats, the two acoustic signals frequency-shifted in relation to one another are presented to the user on two channels, namely on the left ear and on the right ear. In the case of monaural beats, the two frequency-shifted acoustic signals are presented only on one channel.

Monaural or binaural beats such as these can be used to achieve specific training effects for the brain.

European patent EP 2 671 390 B1, corresponding to U.S. Pat. No. 9,426,585, reveals a binaural hearing aid system for producing binaural beats. The two hearing devices in the binaural hearing aid system have sound generators synchronized to one another that each generate an audio signal, one of these audio signals being frequency-shifted in one hearing device. Each of the audio signals is summed with the electrical input signal by way of a summing unit.

### SUMMARY OF THE INVENTION

Against this background, the invention is based on the object of using a hearing system to generate acoustic output signals suitable for producing an agreeable perception of monaural or binaural beats in the user.

The object is achieved according to the invention by a hearing system, in particular a binaural hearing system, having the features of the independent hearing system claim and by a method for producing monaural or binaural beats using such a hearing system. Advantages and preferred arrangements mentioned with regard to the hearing system also apply, mutatis mutandis, to the method, and vice versa.

The hearing system is in particular a hearing aid system that is configured in a user-specific manner and is configured to compensate for a user-specific hearing impairment.

The hearing system has at least one hearing device and preferably, especially in the arrangement as a binaural hearing system, two hearing devices. The at least one hearing device has a primary signal path, containing an input transducer for generating an electrical input signal from a for example acoustic or streamed input signal, containing an in particular digital signal processing unit for processing the electrical input signal and for generating an electrical output signal, and containing an output transducer that takes the electrical output signal as a basis for generating an acoustic output signal. The at least one hearing device moreover has a secondary signal path, containing a respective signal generator for generating a first beat signal. The first beat signal being frequency-shifted in relation to a further signal by a beat frequency in such a way that the effect of the monaural or binaural beat is produced for the user during operation. The at least one hearing device has a summing unit that sums at least the first beat signal with a signal of the primary signal path, with the result that the acoustic output signal is formed by a normal audio signal component and a beat signal component. The signal generator has an associated adjusting apparatus that has an adjustable amplifier element. The adjusting apparatus having an analysis unit for determining an instantaneous signal level of the input signal, and the adjusting apparatus being configured to set a signal level of the beat signal on the basis of the instantaneous signal level.

In the case of a binaural hearing system, each of the two hearing devices forms a communication unit that forms a communication interface via which the two hearing devices alternately communicate with one another and interchange data in a known manner. By way of example, a Bluetooth connection and in particular an NFMI connection is designed for this purpose.

In a hearing system containing two hearing devices, in particular in a binaural hearing system, the second hearing device likewise has a primary signal path containing the components mentioned previously.

In a preferred arrangement, but not imperatively, a second hearing device such as this also has a secondary signal path that likewise contains a signal generator for generating a second beat signal. In this variant embodiment, the two beat signals are frequency-shifted in relation to one another by the beat frequency. The second hearing device in this case also has a summing unit that sums the second beat signal with a signal of the first signal path. This generates an acoustic output signal in the second hearing device too. The acoustic output signal being formed by a normal audio signal component and a beat signal component on the basis of the second beat signal.

Normal audio signal component in the acoustic output signal is understood to mean the signal component that would be obtained without summation of the beat signal.

Furthermore, the signal generator of the second hearing device preferably also has an associated adjusting apparatus containing an adjustable amplifier element and containing an analysis unit for determining the instantaneous signal level of the input signal on the second hearing device. The signal level of the second beat signal is set on the basis of the instantaneous signal level of the input signal of the second hearing device. In a preferred variant, the analysis unit is therefore used to determine a respective instantaneous signal level of the input signal of the respective input transducer in both hearing devices.

Alternatively, there is also the possibility, in particular in a binaural hearing system, of just one hearing device having an analysis unit, or just one hearing device determining the instantaneous signal level and relaying it to the other hearing device via the communication interface.

Overall, the signal-level-dependent setting of the signal level of the beat signal effectively aligns the amplitude and therefore the volume of the respective beat signal with the instantaneous signal level of the input signal. The signal level of the beat signal is therefore set and aligned in particular on the basis of the ambient noise picked up by the at least one input transducer. This produces an agreeable perception of the monaural or binaural beat in the user.

The arrangement of a secondary signal path for the second hearing device is not absolutely essential, not even in the case of binaural hearing devices and/or when binaural beats are being produced.

When monaural beats are being produced, a normal signal component and a frequency-shifted signal component are provided inside the at least one hearing device only, for example, and then lead to perception of the monaural beat. Even when binaural beats are being produced, it is sufficient in a variant embodiment if, for example in the signal generator of the first hearing device, the beat signal is generated on the basis of the input signal merely by way of a frequency shift in the input signal or in signal components of the input signal, for example. The binaural effect is produced for example by the audio signal (acoustic output signal) on the second hearing device and by the frequency-shifted beat signal component (on the basis of the first beat signal) in the acoustic output signal of the first hearing device.

In such variants in particular, a secondary signal path is dispensed with in the second hearing device and/or a frequency-shifted beat signal is generated in the first hearing device only.

However, preference is given to the variant in which both hearing devices have a secondary signal path and a respective signal generator.

Where reference is made to a beat signal in the present case, this is generally understood to mean a signal that contributes, in the acoustic output signal, that is to say the output audio signal, to a signal component that contributes to producing the desired effect of the monaural or binaural beat.

Preferably, an additional, further signal is generated in the respective signal generator in addition to the input signal, the further signal also being referred to as a carrier signal and preferably being independent of the input signal. This is for example a tone (e.g. sinusoidal signal) or in particular narrowband noise.

If no additional signal is generated and the beats are generated only by the normal audio signal and an audio signal component that is frequency-shifted in relation thereto, for example, the normal audio signal and the audio signal component frequency-shifted in relation thereto are the two beat signals that lead to the beats being perceived by the user.

The further signal, in relation to which the first beat signal is shifted by a beat frequency, is in particular the second beat signal generated by the second signal generator. Alternatively, it is the electrical input signal or a signal component derived therefrom. In this alternative variant, the captured ambient sound is thus used to generate the beat signals.

Beat frequency, in the present case, is understood to mean the frequency by which the two beat signals are frequency-shifted in relation to one another. The beat frequency in this instance is typically in the range from 0.5 Hz to 60 Hz, preferably no more than 55 Hz, and often in the range between 3 Hz and 30 Hz.

To generate the frequency-shifted beat signal, at least one signal generator has a frequency shifter, which thus shifts a signal generated or provided in the signal generator (e.g. sinusoidal signal, narrowband noise, signal component from input signal) by the beat frequency and thus generates the frequency-shifted beat signal.

The instantaneous signal level of the input signal is generally a characteristic value for the signal level, that is to say for the amplitude of the acoustic or streamed input signal that is detected by the input transducer. The instantaneous signal level is routinely determined on the basis of an evaluation of the converted electrical input signal. Specifically, the determination of the instantaneous signal level is an estimation. Overall, determination of the instantaneous signal level therefore determines a signal level and thus an amplitude of the instantaneous ambient sound.

In a preferred arrangement, the adjusting apparatus is configured so that the signal level of the beat signal component in the acoustic output signal is 1 dB to 5 dB higher than the signal level of the normal audio signal component in the acoustic output signal. Studies have shown that such a selection is particularly suitable for producing the desired effect of the monaural or binaural beats without running the risk of the (additional) beat signals being perceived as annoying.

Various methods can be used to determine the instantaneous signal level. By way of example, a wideband signal component of the input signal, for example the entire bandwidth of the audible frequency spectrum or only a subband, is evaluated. This involves averaging the signal levels in different frequency bands, for example, and the averaged signal level is considered to be the signal level of the input signal. Alternatively, the frequency band having the highest signal level is used and this maximum signal level is considered to be the signal level of the input signal.

There is also the possibility of stipulating a reference frequency at which the signal level is determined as the reference level and therefore as the instantaneous signal level. By way of example, the reference frequency is a center frequency of a selected frequency band or is the frequency having the highest signal level. The frequency band and/or the reference frequency are firmly predefined, for example, or alternatively dynamically determined on the basis of the respective instantaneous maximum signal level.

According to a preferred arrangement, the reference frequency matches a beat signal frequency of the beat signals, at least within a tolerance range of +/−30 Hz, for example.

Beat signal frequency is initially understood to mean an average frequency of the two beat signals frequency-shifted in relation to one another. If the beat signals consist of a single tone (sinusoidal signal), the frequency of the sinusoidal signal is used. If the respective beat signal has a frequency band, e.g. in the case of narrowband noise, the center frequency of the frequency band is used for the beat signal frequency.

According to a preferred alternative, an offset between the reference frequency and the beat signal frequency is consciously set in order to avoid masking effects. By way of example, the offset is in the range from 50 Hz to 100 Hz. Preferably, the reference frequency is firmly predefined. Alternatively, the reference frequency is dynamically specifically determined on the basis of the frequency of a maximum signal level in each instance, and the beat signal frequency is likewise dynamically adapted.

In a preferred arrangement, at least one of the hearing devices has a synchronizer that is configured to take the instantaneous signal levels determined by the two analysis units as a basis for generating a synchronized setting signal that is used to set the respective signal level of the respective beat signal by way of the respective amplifier element in the respective hearing device. The synchronizer is therefore used to achieve coordinated, synchronized setting of the gain factors for the two beat signals. The synchronizer accomplishes this by comparing the two instantaneous signal levels, for example, and takes the comparison as a basis for deciding what gain factor is set for the respective beat signal.

According to a variant embodiment, there is provision for the two signals to be amplified in such a way that different ratios between the signal level of the respective input signal and the respective beat signal are set for the channels. Specifically, the differences (level interval) between the respective instantaneous signal level and the associated signal level of the associated beat signal are therefore different for the two beat signals. This is advantageous, in particular in the case of highly asymmetric audio sources, if there is thus a dominant lateral acoustic source in a specific hearing situation. The effect achieved by the synchronization and suitable adaptation of the gain factors in the two hearing devices is for example that the signal levels of the beat signals do not diverge too greatly, that is to say are not too different, since a large difference between the signal levels of the beat signals impairs sensory perception of the beats. The synchronizer therefore coordinates the signal levels of the two beat signals with one another.

In a preferred arrangement, the respective instantaneous signal level is recurrently determined within a predefined time interval and the signal level of the beat signal is recurrently set on the basis of the instantaneous signal level. The time interval is preferably no more than 300 ms and in particular no more than 100 ms. This ensures fast and preferably delay-free alignment of the signal level of the beat signals with the instantaneous signal level, resulting in an agreeable sensory perception of the beats.

The signal processing unit used to process the electrical input signal routinely incorporates a signal amplifier that amplifies the, typically already partially conditioned, electrical input signal, specifically normally in a user-specific manner on the basis of the user-specific hearing impairment. The signal amplifier normally also has a compression unit that compresses the signal. According to a preferred arrangement, the summing unit is disposed upstream of this signal amplifier, with the result that the beat signal is thus jointly amplified together with the partially conditioned electrical input signal. This has the advantage that specific advantageous functions of the signal amplifier, for example level limiting for the acoustic output signal delivered by the output transducer, are used for the summed signal.

According to an alternative variant embodiment, the summing unit is disposed downstream of such a signal amplifier.

In a preferred arrangement, the hearing system has a user-specific adjusting element that can be used to manually set the beat signal, in particular the signal level of the beat signal. To this end, the hearing system has a user interface, for example, in particular in the form of a portable device such as a smartphone, on which an application is installed that can be used to make the settings for the respective hearing device. This portable device is part of the hearing system. Alternatively or additionally, operator control elements that can be used to make the setting may also be disposed directly on at least one hearing device.

These manual adjustment options therefore present the user with the opportunity to themselves at least readjust parameters that influence the beats, in order to obtain as agreeable an auditory sensation as possible. The parameters in this instance are in particular the beat frequency and/or the gain factor for the amplifier element, or the signal level of the respective beat signal, in particular the level interval compared with the normal audio signal component in the acoustic output signal.

In a preferred development, the hearing system has a status recognizer, that is to say is designed to automatically recognize a current status of the user. Moreover, the hearing system, in particular the adjusting apparatus, is configured to take the identified current status as a basis for setting the at least one beat signal. Status of the user is understood to mean in particular a current physical state of the user, determined for example by determining the instantaneous pulse of the user and/or by way of acceleration sensors. The current status can be determined either using auxiliary devices or directly using the hearing device too. By way of example, the hearing device may incorporate acceleration sensors, or the hearing device is designed to determine the instantaneous pulse of the hearing device wearer from acoustic signals, e.g. structure-borne sound signals.

In particular, a sleep mode is identified, in particular compared with an awake mode, in which case the parameters are set differently for the different modes. Parameters for the beat signal are in turn understood to mean in particular the beat frequency (that is to say the magnitude of the frequency shift between the beat signals) and/or the signal level. As such, for example an identified sleep mode results in the signal level being reduced compared with an awake mode. Additionally or alternatively, different beat frequencies are set for the awake mode and the sleep mode. This is because it is known that different beat frequencies have different impacts and training effects for the brain.

In a preferred arrangement, the beat frequency is set—for example in the sleep mode—to a range between 35 Hz and 45 Hz and in particular to 40 Hz. Studies have shown that such beat frequencies have particular advantages, especially in the sleep mode.

Other features which are considered as characteristic for the invention are set forth in the appended claims.

Although the invention is illustrated and described herein as embodied in a hearing system, and a method for producing monaural or binaural beats, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE FIGURE

An exemplary embodiment of the invention is explained in more detail below with reference to the FIGURE. The FIGURE shows a simplified block diagram representation of a variant embodiment of a binaural hearing system.

### DETAILED DESCRIPTION OF THE INVENTION

Referring now to the figure of the drawings in detail, there is shown a hearing system 2 in the form of a hearing aid system configured to compensate for a user-specific hearing impairment. This is a binaural hearing system 2 containing two hearing devices 4A, 4B that each communicate with one another in a manner known per se using a wireless communication unit, in particular a Bluetooth communication interface, which is not shown in more detail here. The two hearing devices 4A, 4B are of at least largely identical design. Differences will be discussed explicitly.

A respective hearing device 4A, 4B has one or more respective input transducers 6, in particular microphones, that are used to convert an input signal, in particular an ambient acoustic signal, into an electrical input signal E1, E2. The electrical input signal is first specifically conditioned in a primary signal path 8 and in a signal processing unit 10 in a manner known per se on the basis of a user-specific audiogram and thus in accordance with a user-specific hearing impairment. The conditioned electrical signal is routed as an electrical output signal A1, A2 to an output transducer 12 that converts the electrical output signal into an acoustic output signal aA1, aA2. The output transducer 12 is in particular a loudspeaker.

The signal processing unit 10 has, for example on the input side, a filter bank 14 that is used to break down the electrical input signal E1, E2 into different frequency bands for the subsequent signal processing. After this, there is normally provision for multiple signal processing units 16, 18, for example a beamformer for producing a directionality or a noise suppressor for reducing disruptive ambient noise. An electrical input signal E1, E2 partially conditioned by the signal processing units 16, 18 is then routed to a summing unit 20. Disposed downstream of the summing unit 20 is a signal amplifier 22, typically containing an integrated compression unit. Finally, the signal amplifier 22 is followed, for example, by a synthesizing filter bank 24 that recombines the individual frequency bands and outputs the electrical output signal A1, A2 at the end of this.

Besides the primary signal path 8, a respective hearing device 4A, 4B has a secondary signal path 26. The secondary signal path has a signal generator 28 for generating a beat signal B1, B2. The signal generator 28 is used to provide a further signal, in particular in addition to the electrical input signal E1, E2. The further signal is in particular a signal that is independent of the input signal, specifically a sinusoidal signal or an in particular narrowband noise signal. In addition, there is also the possibility of using a signal component of the electrical input signal E1, E2 picked up by the input transducer 6, that is to say ambient noise, as the basis for the beat signal B1, B2. By way of example, the beat signal B1, B2 is made up of multiple signal components formed on the basis of, or by, the previously mentioned signals (sinusoidal signal, noise signal, ambient noise).

At least one of the two signal generators 28, in the first hearing device 4A in the exemplary embodiment, incorporates a frequency shifter 30 that is used to shift the previously generated signal (e.g. sinusoidal signal, noise signal) by a beat frequency. The two beat signals B1, B2 are therefore two identical signals having the same signal components, which are shifted in relation to one another merely by the beat frequency.

Moreover, a respective hearing device 4A, 4B has a respective analysis unit 32 that is configured to determine, in particular estimate, an instantaneous signal level Lm of the (acoustic) input signal. To this end, the analysis unit 32 suitably evaluates the, possibly partially conditioned, electrical input signal E1, E2 applied upstream of the summing unit 20.

The instantaneous, currently applied signal level Lₘ determined in the process is taken as a basis for determining an instantaneous gain factor used to amplify the respective beat signal B1, B2. A respective amplifier element 34 is provided for amplification purposes. The analysis unit 32 and the amplifier element 34 are parts of an adjusting apparatus 36 that is used to set a respective signal level L_{B} for the beat signal B1, B2.

According to a preferred arrangement as depicted in the figure, at least one of the hearing devices 4A, 4B contains a synchronizer 38 that is connected to the two analysis units 32 and receives therefrom information about the respective signal level Lₘ. This information is relayed from one hearing device 4B to the synchronizer 38 disposed in the other hearing device 4A in particular via the previously described communication interface of the binaural hearing system 2.

The synchronizer 38 evaluates the two instantaneous signal levels Lₘ, in particular compares them, and takes this evaluation as a basis for determining suitable synchronized gain factors for the two beat signals B1, B2. If the instantaneous signal levels Lₘ differ greatly, the synchronizer 38 in particular provides for the differences between the signal levels L_{B} of the beat signals B1, B2 to be limited.

In an alternative, simplified variant embodiment, which is not depicted here, each adjusting apparatus 36 of the two hearing devices 4A, 4B individually determines a respective instantaneous gain factor for the respective beat signal B1, B2. In this variant, the synchronizer 38 and a synchronization are therefore dispensed with.

The respectively amplified beat signal B1, B2 is subsequently routed to the summing unit 20. The resulting compiled signal, which has the partially conditioned input signal E1, E2 and the beat signal B1, B2 as signal components, is then routed to the signal amplifier 22, where it is amplified, and finally provided downstream of the synthesizing filter bank 24 as the outbound electrical output signal A1, A2, which is subsequently converted into the acoustic output signal aA1, aA2 by the output transducer 12.

The respective acoustic output signal aA1, aA2 is therefore made up of a normal audio signal component and a beat signal component. The normal audio signal component is the signal component that would be obtained without the respective signal component of the beat signals. The beat signal components in the acoustic output signal aA1, aA2 are frequency-shifted in relation to one another by the beat frequency and possibly have different amplitudes, but are otherwise identical.

In addition to the normal audio signal, both ears of the user are therefore provided with two (acoustic) beat signals B1, B2 that are offset in relation to one another by the beat frequency, and so, as desired, the binaural beats are perceived by the user.

The instantaneous signal level Lm is detected preferably on an ongoing basis and preferably in time intervals of less than 300 ms, and for example every 100 ms, and the signal level L_{B} of the beat signals B1, B2 is continually aligned on the basis of the detected instantaneous signal level Lₘ. This is preferably carried out in such a way that a higher signal level L_{B} of the beat signal components is produced in the acoustic output signal aA1, aA2 compared with the signal levels Lₘ of the normal audio signal component. Preferably, the signal level L_{B} of the beat signal components is 1 dB to 5 dB, and in particular 3 dB, higher.

In the present case, the electrical input signals are consistently provided with the reference sign E1, E2-irrespective of their processing status—over the entire primary signal path 8.

In the present case, the further (beat) signals provided by the respective signal generator 28 are consistently denoted by the respective reference sign B1, B2 over the entire signal path and therefore in different processing stages.

The previously mentioned units such as filter bank 14, 24, signal processing unit 16, 18, summing unit 20, signal amplifier 22, signal generator 28, frequency shifter 30, analysis unit 32, amplifier element 34 and synchronizer 38 are each electronic devices or groups of electronic circuit components that perform the desired described functions during operation. By way of example, these units are integrated in one or more integrated circuits, microchips and/or ASICS.

The following is a summary list of reference numerals and the corresponding structure used in the above description of the invention:
2 hearing system
4A, B hearing device
6 input transducer
8 primary signal path
10 signal processing unit
12 output transducer
14 filter bank
16 signal processing unit
18 signal processing unit
20 summing unit
22 signal amplifier
24 synthesizing filter bank
26 secondary signal path
28 signal generator
30 frequency shifter
32 analysis unit
34 amplifier element
36 adjusting apparatus
38 synchronizer
E1, E2 electrical input signal
A1, A2 electrical output signal
aA1, aA2 acoustic output signal
B1, B2 beat signal
Lₘ signal level of the input signal
L_{B} signal level of the beat signal

1. A hearing system configured to produce monaural or binaural beats, the hearing system comprising:
at least one hearing device, containing:
a primary signal path having an input transducer for generating an electrical input signal from an acoustic input signal, a signal processor for processing the electrical input signal and for generating an electrical output signal, and an output transducer taking the electrical output signal as a basis for generating an acoustic output signal;
a secondary signal path having a signal generator for generating a first beat signal, the first beat signal being frequency-shifted in relation to a further signal by a beat frequency in such a way that an affect of a monaural beat or binaural beat is produced for a user during operation;
a summing unit for summing at least the first beat signal with a signal of the primary signal path; and
said signal generator including an associated adjusting apparatus having an adjustable amplifier element, said associated adjusting apparatus having an analysis unit for determining an instantaneous signal level of the electrical input signal, and said associated adjusting apparatus being configured to set a signal level of the first beat signal on a basis of the instantaneous signal level.

2. The hearing system according to claim 1, wherein said associated adjusting apparatus is configured so that the signal level of a beat signal component in the acoustic output signal is 1 dB to 5 dB higher than the instantaneous signal level of a normal audio signal component.

3. The hearing system according to claim 1, wherein said associated adjusting apparatus is configured to determine the instantaneous signal level at a reference frequency and in that a beat signal frequency has an offset in relation to the reference frequency.

4. The hearing system according to claim 1, wherein:
said at least one hearing device is one of two hearing devices and each of said hearing devices has said analysis unit and said adjustable amplifier element; and
at least one of said hearing devices has a synchronizer that is configured to take instantaneous signal levels determined by said two analysis units of said hearing devices as a basis for delivering a synchronized setting of signal levels for a respective beat signal to said two adjustable amplifier elements of the hearing devices.

5. The hearing system according to claim 4, wherein a respective said associated adjusting apparatus is configured to recurrently set the signal level) of the beat signal on a basis of the instantaneous signal level of the acoustic output signal within a time interval of no more than 300 ms.

6. The hearing system according to claim 1, wherein said signal processor has a signal amplifier and said summing unit is disposed upstream of said signal amplifier.

7. The hearing system according to claim 1, wherein the hearing system is configured for manual setting of at least one of the first and second beat signals by the user.

8. The hearing system according to claim 1, wherein the hearing system is configured to automatically identify a current status of the user, and in that it is configured to set at least one of the first and second beat signals on a basis of an identified current status.

9. The hearing system according to claim 1, wherein the beat frequency is set to a range from 35 Hz to 45 Hz in an identified sleep mode.

10. A binaural hearing system for producing binaural beats, the binaural hearing system comprising:
hearing devices including at least one first hearing device and a second hearing device, said hearing devices configured for wireless, binaural communication with one another, each of said hearing devices containing:
a primary signal path having an input transducer for generating an electrical input signal, a signal processor for processing the electrical input signal and for generating an electrical output signal, and an output transducer taking the electrical output signal as a basis for generating an acoustic output signal;
a secondary signal path having a respective signal generator for generating a first beat signal in said first hearing device and a second beat signal in said second hearing device, the first and second beat signals being frequency-shifted in relation to one another by a beat frequency such that an effect of a binaural beat is produced for a user during operation;
a summing unit for summing the first and second beat signals with a signal of said primary signal path, with a result that the acoustic output signal is formed by a normal audio signal component and a beat signal component in each instance; and
said respective signal generator having a respective associated adjusting apparatus having an adjustable amplifier element, said respective associated adjusting apparatus of at least one of said hearing devices having an analysis unit for determining an instantaneous signal level of the electrical input signal, and said respective associated adjusting apparatus being configured to set a signal level of the first and second beat signals on a basis of the instantaneous signal level.

11. The hearing system according to claim 10, wherein said associated adjusting apparatus is configured so that the signal level of the beat signal component in the acoustic output signal is 1 dB to 5 dB higher than the instantaneous signal level of the normal audio signal component.

12. The hearing system according to claim 10, wherein said associated adjusting apparatus is configured to determine the instantaneous signal level at a reference frequency and in that a beat signal frequency has an offset in relation to the reference frequency.

13. The hearing system according to claim 10, wherein at least one of said hearing devices has a synchronizer configured to take instantaneous signal levels determined by said two analysis units of said hearing devices as a basis for delivering a synchronized setting of signal levels for a respective one of the first and second beat signals to said two adjustable amplifier elements of said hearing devices.

14. The hearing system according to claim 10, wherein a respective said associated adjusting apparatus is configured to recurrently set the signal level of the beat signal on a basis of the instantaneous signal level of the acoustic output signal within a time interval of no more than 300 ms.

15. The hearing system according to claim 10, wherein said signal processor has a signal amplifier and said summing unit is disposed upstream of said signal amplifier.

16. The hearing system according to claim 10, wherein the hearing system is configured for manual setting of at least one of the first and second beat signals by the user.

17. The hearing system according to claim 10, wherein the hearing system is configured to automatically identify a current status of the user, and configured to set at least one of the first and second beat signals on a basis of an identified current status.

18. The hearing system according to claim 10, wherein the beat frequency is set to a range from 35 Hz to 45 Hz in an identified sleep mode.

19. A method for producing monaural or binaural beats in a hearing system containing at least one hearing device, the at least one hearing device having:
a primary signal path with an input transducer, a signal processor and an output transducer;
a secondary signal path containing a signal generator; and
a summing unit;
the method comprises the steps of:
generating, via the input transducer, an electrical input signal from an acoustic input signal;
processing the electrical input signal in the signal processor which outputs an electrical output signal;
sending the electrical output signal to the output transducer which takes the electrical output signal as a basis for generating an acoustic output signal;
generating via the secondary signal path, a first beat signal, the first beat signal being frequency-shifted in relation to a further signal by a beat frequency such that an effect of a monaural beat or a binaural beat is produced for a user;
summing, via the summing unit, at least the first beat signal with a signal of the primary signal path; and
setting a signal level of the first beat signal on a basis of an instantaneous signal level of the electrical input signal.
